(19) United States
(12) Patent Application Publication
(10) Pub. No.: US 2026/0020981 A1
(43) Pub. Date: Jan. 22, 2026

(54) Wearable Temperature-Reducing Device

(71) Applicant: Jiangsu Huanyi Industrial Co., Ltd., Jiangsu (CN)

(72) Inventor: Kai Zhang, Jiangsu (CN)

(21) Appl. No.: 18/808,011

(22) Filed: Aug. 18, 2024

(30) Foreign Application Priority Data
Jul. 17, 2024 (CN) 202421703820.2

(51) Int. Cl. A61F 7/02; A61F 7/00

(52) U.S. Cl. CPC A61F 7/02; A61F 2007/0011; A61F 2007/0219; A61F 2007/0225; A61F 2007/0247; A61F 2007/0292

(57) ABSTRACT

The present utility model provides a wearable temperature-reducing device, comprising an arcuate body, which comprises a flexible outer wall and a cavity enclosed by the flexible outer wall. The cavity comprises an internal capsule, a phase-change layer located in the internal capsule, and a heat-conducting layer located between the flexible outer wall and the internal capsule, and the arcuate body forms an enclosed space for wearing. The wearable temperature-reducing device of the present application is mainly intended to be worn by humans, and has the advantages of being easy to wear, simple in structure, environmentally friendly, etc.

### CLAIMS OF PRIORITY

This patent application claims priority from:

(1) CN utility model patent application number is 202421703820.2 with a filing date of Jul. 17, 2024 and DAS code of 4E83.

### TECHNICAL FIELD

The present utility model pertains to the wearable field, and more specifically, relates to a wearable temperature-reducing device.

### BACKGROUND

The existing wearable temperature-reducing devices are relatively complex in structure, relatively poor in environmental friendliness, difficult to wear, and relatively low in reuse rate. The wearable temperature-reducing device of the present application is mainly intended to be worn by humans, and has the advantages of being easy to wear, simple in structure, environmentally friendly, etc. The present application designs the wearable temperature-reducing device by applying the principle that a phase-change material will be subject to a phase change at a phase-change temperature to absorb or release heat, without need of complex chemical reactions or energy consumption, and the solution is simple and can be used constantly.

### SUMMARY

The technical problem solved by the present utility model is that the existing wearable temperature-reducing devices are not easy to store and use.

In view of the above concept and other concepts, the present utility model is proposed.

According to one aspect of the present utility model, a wearable temperature-reducing device is provided, comprising:
an arcuate body, which comprises a flexible outer wall and a cavity enclosed by the flexible outer wall, wherein the cavity comprises an internal capsule, a phase-change layer located in the internal capsule, and a heat-conducting layer located between the flexible outer wall and the internal capsule, and the arcuate body forms an enclosed space for wearing.

In one embodiment, a distance between a portion of the flexible outer wall, which portion is close to the enclosed space, and a portion of the internal capsule, which portion is close to the enclosed space, is smaller than that between a portion of the flexible outer wall, which portion is distal from the enclosed space, and a portion of the internal capsule, which portion is distal from the enclosed space.

In one embodiment, the arcuate body has a first end portion and a second end portion, which are opposite to each other.

In one embodiment, an inlet to the enclosed space is formed between the first end portion and the second end portion.

In one embodiment, the enclosed space tapers in a direction toward the inlet.

In one embodiment, the arcuate body has a sealing port, which is used to fill the cavity with the heat-conducting layer.

In one embodiment, the arcuate body has a first end portion and a second end portion, which are opposite to each other, and the first end portion and the second end portion extend away from the sealing port.

In one embodiment, the heat-conducting layer comprises gel.

The present application designs the wearable temperature-reducing device by applying the principle that a phase-change material will be subject to a phase change at a phase-change temperature to absorb or release heat, without the need of complex chemical reactions or energy consumption, the solution is simple and can be used constantly. Wherein, the internal capsule is filled with a phase-change layer made from a phase-change material, and the outer side of the phase-change layer is filled with a heat-conducting layer made from gel.

Additional embodiments of the present utility model can achieve other beneficial technical effects that are not listed one by one, and these other technical effects may be partially described below and can be expected and understood by those skilled in the art after reading the present utility model.

### BRIEF DESCRIPTION OF DRAWINGS

To make the technical solutions in the embodiments of the present utility model clearer, the drawings that need to be used in the description of the embodiments or the prior art will be briefly introduced below. Apparently, the drawings in the following description relate to only some of the embodiments of the present utility model. Without creative labor, those skilled in the art can further obtain other drawings according to these drawings.

FIG. 1 shows a front view of the wearable temperature-reducing device in the present application; and

FIG. 2 shows a cross-sectional view of the wearable temperature-reducing device in FIG. 1.

### DESCRIPTION OF EMBODIMENTS

To make the objectives, technical solutions, and advantages of the embodiments in the present utility model clearer, the technical solutions in the embodiments of the present utility model will be clearly and completely described below in combination with the drawings in the embodiments of the present utility model. Apparently, the described embodiments are some of the embodiments of the present utility model, rather than all of them. In general, the components in the embodiments of the present utility model as described and illustrated in the drawings herein can be arranged and designed in a variety of different configurations.

Accordingly, the following detailed description of the embodiments of the present utility model provided in the drawings is not intended to limit the scope of the present utility model as claimed, but represents only selected embodiments of the present utility model. Based on the embodiments in the present utility model, all other embodiments that are obtained by those skilled in the art without creative labor fall within the scope of protection of the present utility model.

It should be noted that: similar symbols and letters denote similar items in the following drawings, so that once an item is defined in one figure, no further definition or explanation thereof is required in subsequent figures.

In the description of the present utility model, it should be explained that the orientation or positional relationship indicated by the terms such as “up”, “down”, “left”, “right”, “vertical”, “horizontal”, “inner”, and “outer” are based on the orientation or positional relationship shown in the drawings, or the orientation or positional relationship in which the product of the present utility model is placed customarily when used, only for the purpose of facilitating the description of the present utility model and simplifying the description, instead of indicating or implying that the device or element referred to must have a specific orientation, and be constructed and operated in a specific orientation, thus the terms cannot be construed as limitations on the present utility model. Furthermore, the terms such as “first”, “second”, and “third” are used only to distinguish the description, and cannot be understood as indicating or implying the relative importance. In the description of the present utility model, unless otherwise specified, “a plurality” means two or more.

Understandably, the phrases and terms used in the present application are for descriptive purposes, and should not be considered restrictive. Herein, “comprise”, “contain”, “have”, or variations thereof are used to include the items listed thereafter, equivalents thereof, and additional items in an open-ended manner.

The embodiments of the present utility model are described below in detail, and examples of the embodiments are shown in the drawings, wherein the same or similar symbols from the start to the end denote the same or similar elements or elements having the same or similar functions. The embodiments described below with reference to the drawings are exemplary, and are used only for explaining the present utility model, but cannot be construed as limitations on the present utility model.

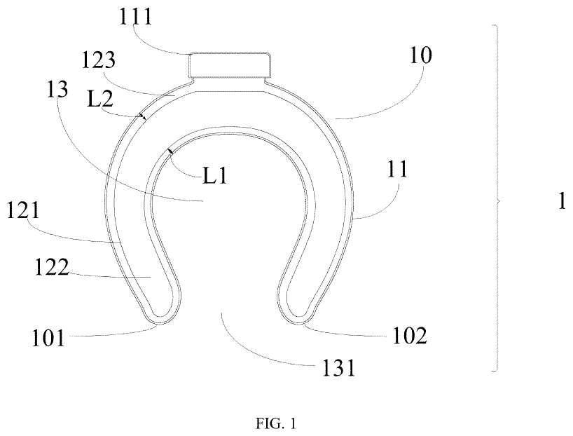

As shown in FIG. 1, a wearable temperature-reducing device 1 comprises: an arcuate body 10, which is flexible, to facilitate a user bending the same. The arcuate body 10 comprises a flexible outer wall 11 and a cavity 12 enclosed by the flexible outer wall 11. The flexible outer wall 11 can be composed of a high molecular material that has elastic deformation and smooth feel, such as thermoplastic polyurethane elastomers (TUP), to facilitate the user bending the same. The cavity 12 comprises an internal capsule 121, a phase-change layer 122 located in the internal capsule 121, and a heat-conducting layer 123 located between the flexible outer wall 11 and the internal capsule 121. The arcuate body 10 forms an enclosed space 13 for wearing. The enclosed space 13 can be presented in a U shape, a circular shape, or an oval shape. When the wearable temperature-reducing device 1 is in a state of use, the enclosed space 13 is intended to be worn around the neck of the user or other parts of the body. The use of the U-shaped, circular-shaped, or oval-shaped enclosed space 13 facilitates the wearable temperature-reducing device 1 fitting the skin of the body of the user.

As shown in FIG. 1, the heat-conducting layer 123 and the phase-change layer 122 are not in contact with each other, and are separated by the internal capsule 121 to avoid mixing the materials of the heat-conducting layer 123 and the phase-change layer 122 or prevent them from chemical reactions. The phase-change layer 122 has a phase-change material, and the heat-conducting layer 123 has gel. Regarding the gel, under appropriate conditions, high molecular solution or sol at a certain concentration has a gradually increasing viscosity, and finally loses its fluidity, so that the entire system becomes an clastic semi-solid, which has a uniform appearance and maintains a certain morphology, and this elastic semi-solid is called gel. The phase-change material (PCM) is a material that can change its physical form and provide latent heat, and the process of transforming its physical properties is called phase-change process, in which the phase-change material will absorb or release a large amount of latent heat. The phase-change material used in the present application is in a liquid state above 28° C. and is in a solid state below 28° C. Through the phase change of the phase-change material between the liquid state and the solid state, the wearable temperature-reducing device 1 can provide a temperature-reducing effect for the user. Of course, the present application does not impose the limitation that the phase-change material as used has a phase-change temperature of 28° C., and other phase-change temperatures close to the human body temperature are also within the optional range.

As shown in FIG. 1, the distance L1 between a portion of the flexible outer wall 11, which portion is close to the enclosed space 13, and a portion of the internal capsule 121, which portion is close to the enclosed space 13, is smaller than the distance L2 between a portion of the flexible outer wall 11, which portion is distal from the enclosed space 13, and a portion of the internal capsule 121, which portion is distal from the enclosed space 13. Herein, the distance refers to straight-line distance. The arrangement mainly facilitates the internal capsule 121 getting closer to the enclosed space 13, and further closer to the skin of the body of the user, thereby ending up with a more sensitive temperature response speed.

As shown in FIG. 1, the flexible outer wall 11 has a sealing port 111, which is used to fill the flexible outer wall 11 with gel to act as a heat-conducting layer 123. The sealing port 111 can be sealed after filling with gel to avoid improper use from leading to leakage of the gel.

As shown in FIG. 1, the arcuate body 10 has a first end portion 101 and a second end portion 102, which are opposite to each other, and the first end portion 101 and the second end portion 102 extend away from the sealing port 111. The first end portion 101 and the second end portion 102 form an inlet 131 of the enclosed space 13, and the enclosed space 13 tapers in the direction toward the inlet 131. The construction of the enclosed space 13 gradually narrowing in the direction toward the inlet 131 is mainly to ensure the firmness of the wearable temperature-reducing device 1 when it is worn on the body of the user. The flexible arcuate body 10 and the narrowed inlet 131 avoid separation of the body of the user from the wearable temperature-reducing device 1. The first end portion 101 and the second end portion 102, which are flexible, can provide a counter-acting force when the body of the user exerts a squeezing force on the wearable temperature-reducing device 1, thereby creating an obstacle for the body of the user to detach from the enclosed space 13 of the wearable temperature-reducing device 1.

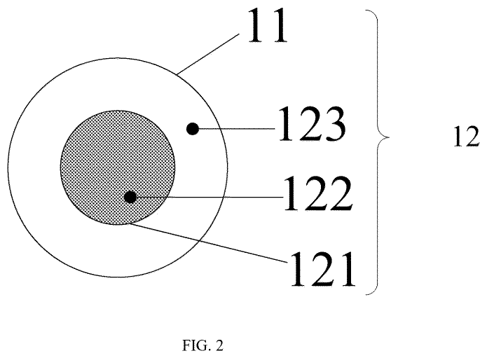

As shown in FIG. 2, the internal capsule 121 is located in the flexible outer wall 11. The cavity comprises the internal capsule 121, the phase-change layer 122 located in the internal capsule 121, and the heat-conducting layer 123 located between the flexible outer wall 11 and the internal capsule 121. The heat-conducting layer 123 and the phase-change layer 122 are not in contact with each other, and are separated by the internal capsule 121, so as to avoid mixing the materials of the heat-conducting layer 123 and the phase-change layer 122 or prevent them from chemical reactions. The phase-change layer 122 has an cross section that is in an approximately circular shape and is nested within the heat-conducting layer 123, i.e., the heat-conducting layer 123 is located around the periphery of the phase-change layer 122, and together with the phase-change layer 122, forms a nested structure.

The wearable temperature-reducing device 1 is used in the following method.

When he or she needs to wear the device, the user can pull the first end portion 101 apart from the second end portion 102 to expand the width of the inlet 131, place a wearing part into the enclosed space 13, and bring the wearing part into contact with the flexible outer wall 11. Since the temperature of the gel in the heat-conducting layer 13 is lower than the temperature of the human skin, the human body transmits heat to the heat-conducting layer 13 to realize the temperature reduction of the human body while raising the overall temperature of the heat-conducting layer 13. The phase-change layer 122 can undergo a phase change to absorb heat from the heat-conducting layer 13. When the phase-change layer 122 is at a phase-change temperature (e.g., 28° C.) of the phase-change material inside the internal capsule 121, the phase-change material absorbs the heat from the gel to undergo a phase change, thereby reducing the temperature of the gel.

The wearable temperature-reducing device of the present application is mainly intended to be worn by humans, and has the advantages of being easy to wear, simple in structure, environmentally friendly, etc. The present application designs the wearable temperature-reducing device by applying the principle that a phase-change material will be subject to a phase change at a phase-change temperature to absorb or release heat, without need of complex chemical reactions or energy consumption, and the solution is simple and can be used constantly.

The above content only relates to preferred embodiments of the present utility model, and is not used to limit the present utility model. Any modifications, equivalent substitutions, and improvements that are within the spirit and principle of the present utility model shall fall within the scope of protection of the present utility model.

1. A wearable temperature-reducing device, comprising:
an arcuate body, which comprises a flexible outer wall and a cavity enclosed by the flexible outer wall, wherein the cavity comprises an internal capsule, a phase-change layer located in the internal capsule, and a heat-conducting layer located between the flexible outer wall and the internal capsule, and the arcuate body forms an enclosed space for wearing.

2. The wearable temperature-reducing device of claim 1, wherein a distance between a portion of the flexible outer wall, which portion is close to the enclosed space, and a portion of the internal capsule, which portion is close to the enclosed space, is smaller than that between a portion of the flexible outer wall, which portion is distal from the enclosed space, and a portion of the internal capsule, which portion is distal from the enclosed space.

3. The wearable temperature-reducing device of claim 1, wherein the arcuate body has a first end portion and a second end portion, which are opposite to each other.

4. The wearable temperature-reducing device of claim 3, wherein an inlet to the enclosed space is formed between the first end portion and the second end portion.

5. The wearable temperature-reducing device of claim 4, wherein the enclosed space tapers in a direction toward the inlet.

6. The wearable temperature-reducing device of claim 1, wherein the arcuate body has a sealing port, which is used to fill the cavity with the heat-conducting layer.

7. The wearable temperature-reducing device of claim 6, wherein the arcuate body has a first end portion and a second end portion, which are opposite to each other, and the first end portion and the second end portion extend away from the sealing port.

8. The wearable temperature-reducing device of claim 1, wherein the heat-conducting layer comprises gel.
